(19) United States
(12) Patent Application Publication
(10) Pub. No.: US 2025/0255731 A1
(43) Pub. Date: Aug. 14, 2025

(54) LATTICE STRUCTURE IMPLANT AND METHODS FOR MAKING THE SAME

(71) Applicant: BFM Holdings, LLC, Austin, TX (US)

(72) Inventors: Rylan Reed, Austin, TX (US); Ali Kiapour, Newton, MA (US); Alexander Rohlfing, St. Louis, MO (US); Joshua Clayton Lyon, Spicewood, TX (US)

(21) Appl. No.: 19/049,467

(22) Filed: Feb. 10, 2025

Related U.S. Application Data
(60) Provisional application No. 63/551,396, filed on Feb. 8, 2024.

(51) Int. Cl. A61F 2/44; A61F 2/30

(52) U.S. Cl. CPC A61F 2/4465; A61F 2/447; A61F 2002/30593; A61F 2002/30777; A61F 2002/3079; A61F 2002/30841

(57) ABSTRACT

An implant is provided. The implant may include a top surface, a bottom surface opposite the top surface, and a body positioned between the top surface and the bottom surface. The top surface may include a first surface lattice, and the bottom surface may include a second surface lattice. Additionally, the body may substantially occupy a volume between the top and bottom surfaces, further defining one or more side surfaces of the implant. Moreover, the body, the first surface lattice, and the second surface lattice may include a plurality of lattice structures.

### CROSS-REFERENCE TO RELATED APPLICATIONS

This Application claims priority to U.S. Provisional Patent Application Ser. No. 63/551,396, filed on Feb. 8, 2024, entitled “LATTICE STRUCTURE IMPLANT SYSTEM AND METHODS FOR MAKING THE SAME,” currently pending, the entire disclosure of which is incorporated herein by reference.

### BACKGROUND

Spinal implants are used to stabilize the spine, alleviate pain, and restore function in cases of back pain, deformity, and disorders such as scoliosis, kyphosis, degenerative disc disease, and fractures. These implants can be categorized as fusion and non-fusion types. Whereas fusion implants promote the joining of vertebrae into a single unity for stability, non-fusion implants support the spine without restricting movement, preserving flexibility. Surgical techniques and approaches vary depending on the type of implant. An Anterior Lumbar Interbody Fusion (“ALIF”) approach includes removing a damaged disc in the lumbar spine through an incision in the abdomen. In contrast, a Transforaminal Lumbar Interbody Fusion (“TLIF”) is performed through a posterior approach, where the damaged disc is removed through an incision at the back. Further, an Anterior Cervical Discectomy and Fusion (“ACDF”) surgery may remove the damaged or herniated disc in the neck and fuse adjacent vertebrae to relive pain, stabilize the spine, and prevent further nerve compression. For the ALIF, TLIF, and ACDF approaches, a spinal implant may be inserted to provide stability, promote fusion, and maintain proper alignment.

Spinal implants may include rods, plates, screw, and interbody cages. Current interbody cages may be cylindrical or tubular in shape and filled with a bone graft. The interbody cages may then be inserted between two adjacent vertebrae to promote bone growth over time, thus fusing the two adjacent vertebrae. Although widely used in the field, current interbody cages present many complications and limitations. First, current interbody cages may not be patient specific nor biomechanically compatible with the spine, leading to complications such as subsidence (i.e., the cage sinking into the spine), misalignment, and inadequate bone growth. Second, the materials used in the interbody cages may not provide optimal strength, flexibility, or proper mechanical properties which may result in long-term outcomes. Moreover, interbody cages may not mimic the natural curvature and movement of the spine. Further, current interbody cages may include a unit cell lattice structure which inhibits optimal osteogenesis. Thus, there is a need for a spinal implant that is not only biocompatible but also biomechanically compatible, where the spinal implant enhances stability, encourages osseointegration, complements patient anatomy, and provides optimal mechanical properties to promote effective healing and minimize the need for future revision surgeries.

### SUMMARY

In accordance with some embodiments of the present disclosure, a lattice structure implant including one or more lattice structures is provided. The implant and methods disclosed herein provide for the ability to develop a lattice structure implant that meets patient-specific needs and overcomes the shortcomings related to current interbody cages.

In one aspect, an implant is provided. The implant may include a top surface having a first surface lattice and a bottom surface opposite the first surface. In some instances, the bottom surface may include a second surface lattice. Additionally, the implant may include a body positioned between the top and bottom surfaces. In some instances, the body may include a first plurality of lattice structures. Moreover, the body may at least partially define one or more side surfaces of the implant. In some instances, the first surface lattice, the second surface lattice, and the body may be integrally formed.

In some instances, the first surface lattice, the second surface lattice, and the body are at least partially smooth. In other instances, the first surface lattice, the second surface lattice, and the body may be at least partially coarse. In yet other instances, the implant may further comprise a first surface lattice comprising a second plurality of lattice structures, and a second surface lattice comprising a third plurality of lattice structures.

In some instances, each lattice structure of the first plurality of lattice structures may comprise one or more lattice struts that converge at one or more lattice junctures. In such instances, the first plurality of lattice structures may include one or more geometric patterns formed from the convergence of the one or more lattice struts at the one or more lattice junctures.

In other instances, the first plurality of lattice structures may be formed from a series of repeated lattices structures mirrored over at least one of a vertical axis and a horizontal axis. In yet other instances, the implant may be symmetrical with respect to at least one of a horizontal axis and a vertical axis.

In some instances, the implant may be configured to be inserted into a spine of a user to provide stability, promote osseointegration, and maintain proper spinal alignment. In other instances, the implant may be at least partially formed from at least one of a Titanium alloy and nitinol.

In another aspect, an implant to provide stability and promote osseointegration is provided. The implant may include a top surface having a first surface lattice and a bottom surface, opposite the top surface, having a second surface lattice. The implant may also include one or more side surfaces. In certain instances, the one or more side surfaces may include a bore configured to receive a screw. Additionally, the implant may include a substantially hollow body that is disposed between the top and bottom surfaces. In some instances, the body may at least partially define the one or more side surfaces. In other instances, the first surface lattice and the second surface lattice at least partially overlay the body about the top surface and the bottom surface, respectively.

In yet other instances, the implant may further comprise a first solid perimeter that defines an outer periphery of the top surface. Additionally, the implant may comprise a similar second solid perimeter defining an outer periphery of the bottom surface. In such instances, the first and second solid perimeter may bound the first and second surface lattices, respectively.

In some instances, each of the first surface lattice and the second surface lattice may comprise a plurality of microlattice structures. In such instances, the plurality of microlattice structures may be substantially thinner than the plurality of lattice structures.

In other instances, the body may couple to the first surface lattice and the second surface lattice. In yet other instances, the body may be integrally formed with the first surface lattice and the second surface lattice.

In some instances, the implant may further comprise one or more keels configured to couple the implant to one or more surroundings bones upon insertion.

In yet another aspect, a spinal implant is provided. The spinal implant may include a top surface including a first surface lattice and a bottom surface, opposite the top surface, including a second surface lattice. Further, the spinal implant may include a substantially hollow body including an open framework formed from a plurality of lattice structures. In certain instances, the body may define one or more side surfaces of the spinal implant. In some instances, the first surface lattice and the second surface lattice may at least partially overlay the body about the top surface and the bottom surface, respectively. Moreover, the spinal implant may include an annular channel that extends linearly from the top surface to at least a center of the spinal implant. In some instances, the annular channel may define a space between the top surface and the center of the spinal implant that is free of lattice structures.

In some instances, the annular channel may be configured to house a bone graft. In other instances, the annular channel may extend from the top surface to the bottom surface.

These and other aspects and advantages of the present invention will become apparent to those skilled in the art after considering the following detailed description in connection with the accompanying drawings.

### BRIEF DESCRIPTION OF THE DRAWINGS

FIG. 1 is a perspective view of a lattice structure implant according to a first embodiment.

FIG. 2 is a planar top view of the lattice structure implant of FIG. 1.

FIG. 3 is an isometric side view of the lattice structure implant of FIG. 1.

FIG. 4 is a perspective view of a lattice structure implant constructed following the teachings of the present disclosure, according to a second embodiment.

FIG. 5 is a planar top view of the lattice structure implant of FIG. 4.

FIG. 6 is perspective view of a lattice structure implant constructed following the teachings of the present disclosure, according to a third embodiment.

FIG. 7 is perspective view of a lattice structure implant constructed following the teachings of the present disclosure, according to a fourth embodiment.

FIG. 8 is perspective view of a lattice structure implant constructed following the teachings of the present disclosure, according to a fifth embodiment.

FIG. 9 is an isometric front view of a lattice structure implant constructed following the teachings of the present disclosure, the lattice structure implant configured to include keels, according to a sixth embodiment.

FIG. 10 is a perspective view of a lattice structure implant constructed following the teachings of the present disclosure, the lattice structure implant configured to include keels, according to a seventh embodiment.

FIG. 11 is an isometric view of a wire graph.

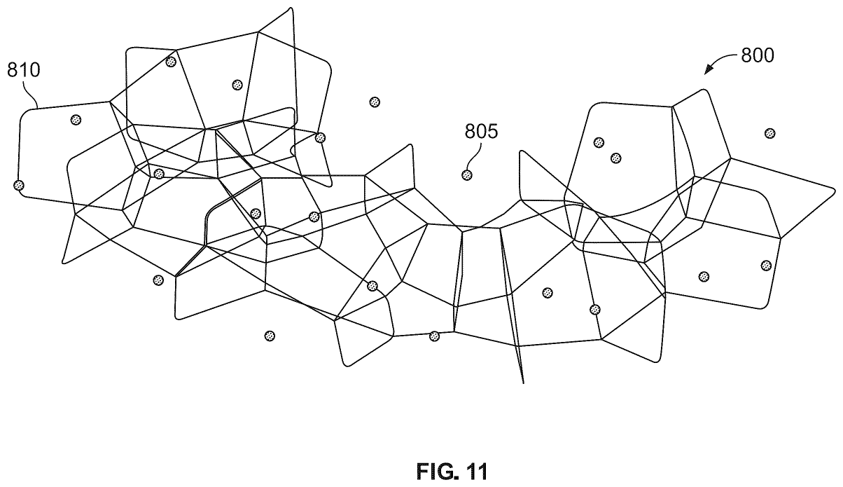

FIG. 12 is a perspective view of a one-fourth portion of a body lattice.

FIG. 13 is a perspective view a body structure formed by the mirror of the one fourth portion of FIG. 1.

FIG. 14 is a perspective view of the body structure from FIG. 13 having varying thickness.

FIG. 15 is a perspective view of a surface lattice.

FIG. 16 is a perspective view of a lattice structure implant constructed following the teachings of the present disclosure, the lattice structure implant having a surface roughness, according to an eighth embodiment.

FIG. 17 is a perspective view of a lattice structure implant constructed following the teachings of the present disclosure, the lattice structure implant including serrations, according to a ninth embodiment.

FIG. 18 is a perspective view of a lattice structure implant constructed following the teachings of the present disclosure, the lattice structure implant having a torus structure, according to a tenth embodiment.

FIG. 19 is a stress distribution diagram of a lattice structure implant according to various embodiments.

FIG. 20 is a perspective view of a lattice structure implant constructed following the teachings of the present disclosure, the lattice structure implant including a lattice covering over a window of an annular channel, according to an eleventh embodiment.

FIG. 21 is a perspective view of a lattice structure implant constructed following the teachings of the present disclosure, according to a twelfth embodiment.

FIG. 22 is a perspective view of a body representing an offset volume.

FIG. 23 is an isometric side view of a configuration between the lattice structure implant and body of FIGS. 21 and 22, respectively.

FIG. 24 is a planar top view of the configuration of FIG. 23.

While the disclosure is susceptible to various modifications and alternative forms, a specific embodiment thereof is shown by way of example in the drawings and will herein be described in detail. It should be understood, however, that the drawings and detailed description presented herein are not intended to limit the disclosure to the particular embodiment disclosed, but to the contrary, the intention is to cover all modifications, equivalents, and alternatives falling within the spirit and scope of the present disclosure as defined by the appended claims.

### DETAILED DESCRIPTION

Before any embodiments are described in detail, it is to be understood that the disclosure is not limited in its application to the details of construction and the arrangement of components set forth in the following description or illustrated in the following drawings, which is limited only by the claims that follow the present disclosure. The disclosure is capable of other embodiments, and of being practiced, or of being carried out, in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Unless specified or limited otherwise, the terms “mounted,” “connected,” “supported,” and “coupled” and variations thereof are used broadly and encompass both direct and indirect mountings, connections, supports, and couplings. Further, “connected” and “coupled” are not restricted to physical or mechanical connections or couplings.

Briefly described, the following discussion is presented to enable a person skilled in the art to make and use embodiments of the system. Various modifications to the illustrated embodiments will be readily apparent to those skilled in the art, and the generic principles herein can be applied to other embodiments and applications without departing from embodiments of the system. Thus, embodiments of the invention are not intended to be limited to embodiments shown but are to be accorded the widest scope consistent with the principles and features disclosed herein. The following detailed description is to be read with reference to the figures, in which like elements in different figures may include differing or like reference numerals. The figures, which are not necessarily to scale, depict selected embodiments and are not intended to limit the scope of embodiments of the system.

Additionally, while the following discussion may describe features associated with specific devices or embodiments, it is understood that additional devices and/or features can be used with the described systems and methods, and that the discussed devices and features are used to provide examples of possible embodiments, without being limited.

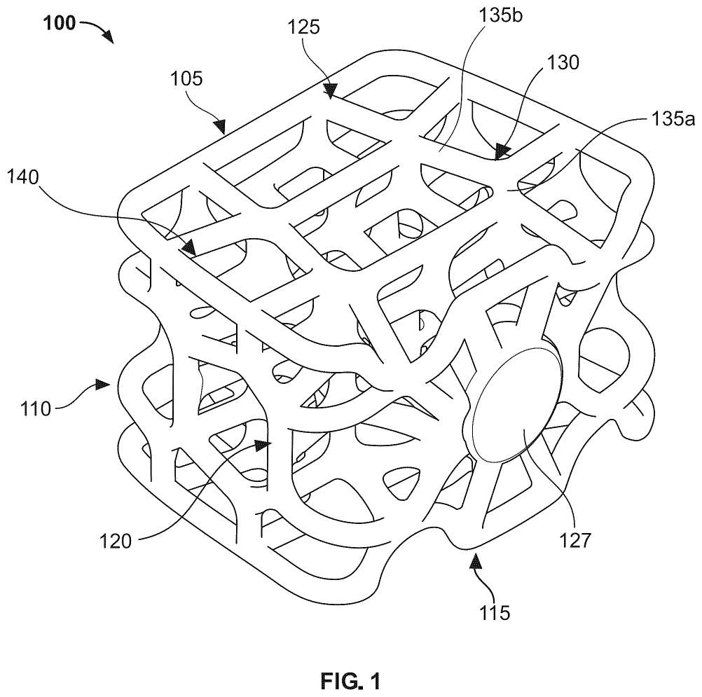

Referring to FIGS. 1-3, an example of a lattice structure implant 100 is provided. Generally, the lattice structure implant 100 may be inserted into a patient's body to support, stabilize, and facilitate healing of the spine. In various instances, the lattice structure implant 100 may be a corpectomy device. The lattice structure implant 100 maybe inserted in place of a damaged or removed disc. In particular, the lattice structure implant 100 may be positioned between two adjacent vertebrae to provide structural support, maintain proper alignment, promote fusion, restore disc height, reduce movement, and the like. In various instances, the lattice structure implant 100 may be inserted into a patient during an Anterior Cervical Discectomy and Fusion (“ACDF”) surgical process. Although, in other instances, the lattice structure implant 100 may support various spinal fusion techniques including, but not limited to, anterior lumbar interbody fusion (“ALIF”) and transforaminal lumbar interbody fusion (“TLIF”). Although described herein with reference to humans, it is to be understood that the lattice structure implant 100 is similarly applicable for use in other animals. Further, the lattice structure implant 100 is not so limited as solely being applicable for spinal fusions. In various instances, the lattice structure implant 100 may also be configured to support knee replacements, hip replacements, and the like. Moreover, the lattice structure implant 100 may be substantially formed from a material that is suitable for biomedical and industrial applications such as Titanium alloy. In other instances, the lattice structure implant 100 may be formed from a combination of biocompatible materials. For example, in some instances, the lattice structure implant 100 may be include nitinol. In various, the lattice structure implant 100 may be formed from one or more biocompatible materials that impart the lattice structure implant 100 with properties such as elasticity and memory.

The lattice structure implant 100 may include a top surface 105, one or more side surfaces 110, and a bottom surface 115 that is opposite the top surface 105. Further, between the top surface 105 and bottom surface 11, the lattice structure implant 100 may have a volume. In some instances, the volume may be substantially hollow. In various instances, the lattice structure implant 100 may include at least four side surfaces 110, each of the four side surface 110 coupling to the top and bottom surfaces 105, 115. In some instances, edges of any of the top surface 105, side surfaces 110, and bottom surface 115 may taper inwardly or outwardly. For example, an edge of a first side surface 110 may taper inwardly towards the top surface 105. In such instances, the taper may be gradual and linear. Alternatively, the taper may be abrupt and curved. In some instances, the lattice structure implant 100 may be substantially cuboidal, trapezoidal, or otherwise include relatively angular features. In other instances, the lattice structure implant 100 may be substantially rounded. For example, rather than having distinct top and bottom surfaces 105, 115 and side surfaces 110, the lattice structure implant 100 may be spherical. In yet other instances, the lattice structure implant 100 may be substantially cylindrical.

As best illustrated in FIG. 1, the lattice structure implant 100 may comprise a body 120 and one or more surface lattices 125. The body 120 may substantially fill a volume of the lattice structure implant 100, where the volume is a space between the top surface 105, the side surfaces 110, and the bottom surface 115. In some instances, the top surface 105, the side surfaces 110, and the bottom surface 115 may bound the body 120. Generally, each the top surface 105 and the bottom surface may include a surface lattice 125. In some instances, the surface lattices 125 of the top and bottom surfaces 105, 115 may be substantially similar. In other instances, the surface lattices 125 of the top and bottom surfaces 105, 115 may be substantially different. In various instances, the surface lattices 125 may substantially extend through an entire length and width of each the top and bottom surfaces 105, 115, thus defining the top and bottom surfaces 105, 115. Moreover, the body 120 and the one or more surface lattice 125 may be substantially hollow. That is, the body 120 and the one or more surface lattices 125 may include a combination of solid and porous elements.

In various instances, the top surface 105, the side surface 110, the bottom surface 115, the body 120, and the surface lattices 125 may be connected. In some instances, the top surface 105, the side surface 110, the bottom surface 115, the body 120, and the surface lattices 125 may be integrally formed. In various instances, the body 120 may substantially define the size and volume of the lattice structure implant 100. Further, the body 120 may substantially define the side surfaces 110 of the lattice structure implant 100. Similarly, the surface lattices 125 may substantially define top and bottom surfaces 105, 115 of the lattice structure implant 100. In some instances, a shape of the lattice structure implant 100 may be at least partially based on the body 120. In other instances, a shape of the lattice structure implant 100 may be based on patient anatomy, where the body 120 may be configured to impart designed and patient specific mechanical properties for providing support, alignment, and osseointegration.

Continuing to reference FIG. 1, in some instances, the lattice structure implant 100 may include a substantially solid element positioned relatively central to at least one side surface 110. For example, as illustrated in FIG. 1, a rounded, substantially solid element 127 may be medially disposed about one side surface 110. In some instances, an opposing side surface 110 may include a substantially similar solid element (not illustrated). In other instances, the solid element 127 may be substantially angular. In various instances, the solid element 127 may be an attachment mechanism configured to receive additional hardware for further securing the lattice structure implant 100 to the surrounding environment. For example, in some instances, the solid element 127 may be a bore configured to receive a screw or a rod.

Each the body 120 and the surface lattices 125 may include one or more porous lattice structures 130 configured to promote osseointegration. In some instances, when the lattice structure implant 100 is inserted into a spine of a patient, the open and porous structure of the lattice structures 130 may enhance affixation of the lattice structure implant 100 by providing a scaffolding for the vertebrae to grow therethrough. In various instances, the lattice structures 130 may directly contact the one or more bones designated for anchoring the lattice structure implant 100. In some instances, the lattice structures 130 may be treated with an osseo-integration coating to promote direct bonding between the lattice structure implant 100 and the surrounding bone tissue. Although the lattice structures 130 may be generally smooth, in some instances, the lattice structures 130 may be relatively coarse to encourage cellular adhesion between the lattice structure implant 100 and bony areas.

Generally, a plurality of lattice structures 130 may form the body 120. Similarly, a plurality of lattice structures 130 may form the surface lattices 125. In various instances the body 120 and the surface lattices 125 may be integrally formed. That is, the body 120 and the surface latices 125 may comprise the same lattice structures 130. Alternatively, the lattice structures 130 of the body 120 may be different than the lattice structures 130 of the surface lattices 125. For example, the shape, size, and/or distance between lattice structures 130 of the body 120 may be smaller or larger than the shape, size, and/or distance between the lattice structures 130 of the surface lattices 125. Moreover, the lattice structures 130 of the body 120 may be denser than the lattice structure of the surface lattices 125.

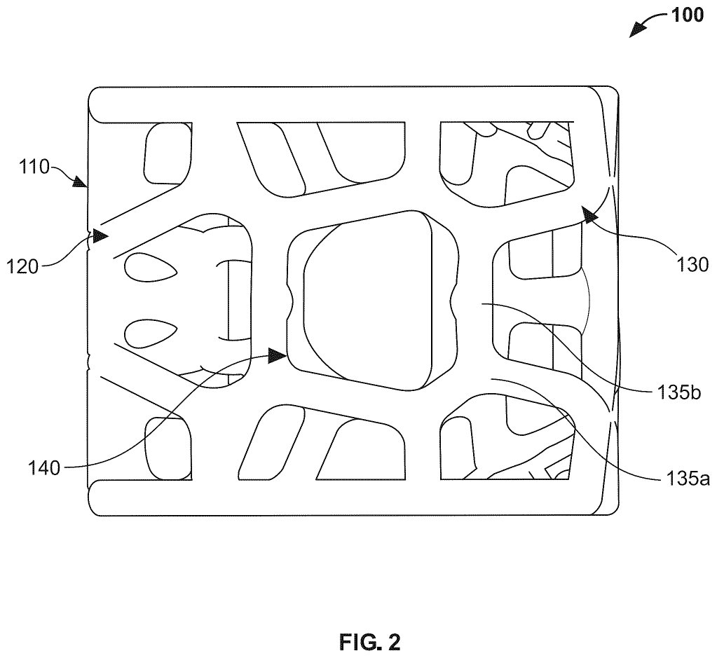

As best illustrated in FIGS. 1 and 2, a body 120 may comprise a plurality of lattice structures 130. In various instances, a lattice structure 130 may include a framework consisting of a series of intersecting elements, thus forming a grid-like or network pattern. The lattice structure 130 may comprise a plurality of lattice junctures 135a connected by a plurality of lattice struts 135b. Generally, lattice junctures 135a may be points or nodes at which one or more lattice struts 135b may converge, forming lattice structures 130. For example, two lattice struts 135b may converge at a single lattice juncture 135a. In another example, three lattice struts 135b may converge at a single lattice juncture 135a. In yet another example, four lattice struts 135b may converge at a single lattice juncture 135a.

To form the lattice structure 130 in a body 120, a plurality of lattice junctures 135a may be dispersed throughout a volume, where the volume is the space at which the body 120 may occupy in the lattice structure implant 100. In various instances, the volume is the space between the top surface 105, side surfaces 110, and bottom surface 115. Each of the lattice junctures 135a may be connected by one or more lattice struts 135b, where the interconnections between the lattice struts 135b at each of the lattice junctures 135a substantially forms the overall grid-like or network pattern of the lattice structures 130. In various instances, the positioning of the lattice junctures 135a may at least partially determine a 3-dimmensional shape of the body 120. In such instances, the 3-dimmensional shape of the lattice structure implant 100 may be at least partially based on the body 120 formed from the plurality of lattice structures 130. For example, the lattice junctures 135a may be positioned at points within the volume with respect to an X-axis, a Y-axis, and a Z-axis. As such, the lattice struts 135b may move with respect to the X-axis, the Y-axis, and the Z-axis to connect each of the lattice junctures 135a, forming an overall 3-dimmensional body 120 of the lattice structure implant 100. In some instances, the lattice structure implant 100 may be imparted with an overall 3-dimmensional shape that is substantially similar to a cube, a tetrahedron, a rectangular prism, a trapezoidal prism, and the like. Alternatively, the lattice structure implant 100 may be imparted with an overall 3-dimmensional shape that is substantially rounded.

As lattice struts 135b connect to one more lattice junctures 135a, a geometric pattern 140 of a lattice structure 130 may be formed. The geometric pattern 140 may be at least partially based on the surrounding lattice junctures 135a and lattice struts 135b. In particular, in some instances, the positioning, amount, and distance between the lattice junctures 135a and the curvature or linearity of the lattice struts 135b connecting each lattice junctures 135a may at least partially influence the geometric pattern 140. For example, four lattice junctures 135a may be connected via four substantially linear lattice struts 135b, thus forming four convergence areas. Accordingly, the lattice structure 130 formed from the four lattice junctures 135a and the four lattice struts 135b may be imparted with a geometric pattern 140 that is substantially rectangular. In some instances, a lattice structure 130 may be imparted with a geometric pattern 140 that is substantially trapezoidal or rectilinear. In another example, the lattice structure 130 may be imparted with a geometric shape 140 that is substantially rounded, circular, and the like. For example, a lattice strut 135b connecting two lattice junctures 135a may have a substantially arcuate, bowed, or curved shape. As such, the arced lattice strut 135b may provide a rounded aspect to the formed geometric pattern 140. In yet another example, the lattice structure 130 may be imparted with a geometric pattern 140 that is substantially amorphous. In some instances, a lattice strut 135b connecting two lattice junctures 135a may be imparted with a substantially hour-glass shape. In other instances, a lattice strut 135b connecting two lattice junctures 135a may be imparted with a substantially sinusoidal shape, such that the lattice strut 135b forms peaks and troughs between the two lattice junctures 135a. In various instances, the lattice structure 130 includes various geometric patterns 140.

As previously discussed, the body 120 may comprise a plurality of lattice structures 130. In some instances, the plurality of lattice structures 130 may be substantially identical to each other. For example, a first lattice structure 130 may be formed, and a second lattice structure 130 may be formed by substantially duplicating and translating the positioning, shape, and elements of the first lattice structure 130 (e.g., the lattice junctures and struts 135a, 135b), thus forming a substantially identical second lattice structure 130. In other instances, a first lattice structure 130 may be formed, and a second lattice structure 130 may be formed by reflecting, rotating, or translating elements of the first lattice structure 130 (e.g., the lattice junctures and struts 135a, 135b) over the X-axis, the Y-axis, and/or the Z-axis. In such instances, the second lattice structure 130 may be substantially similar to the first lattice structure 130. Generally, the body 120 may comprise a plurality of lattice structures 130, where each lattice structure 130 of the plurality of lattice structures 130 includes repeated elements that are either substantially identical or substantially similar (e.g., mirrored over the X-axis, Y-axis, or Z-axis). In some instances, the lattice structure implant 100 may be substantially symmetrical with respect to a horizontal axis (e.g., the X-axis). In other instances, the lattice structure implant 100 may be substantially symmetrical with respect to a vertical axis (e.g., the Y-axis). Alternatively, the lattice structure 130 may be substantially complex, consisting of non-repetitive or irregular configurations of the lattice junctures and struts 135a, 135b.

It is to be understood that the surface lattices 125 may comprise substantially similar components as the body 120. In particular, the surface lattices 125 may likewise include a plurality of lattice structure 130 including a plurality of lattice junctures 135a and a plurality of lattice struts 135b, where the lattice struts 135b are configured to connect to lattice junctures 135a, resulting in a geometric pattern 140 of the lattice strut. In various instances, the same lattice junctures 135a and lattice struts 135b may be used for forming the body 120 and the surface lattices 125. In some instances, the body 120 and the surface lattices 125 may be integrally formed. That is, the body 120 and the surface lattices 125 may be formed from the same plurality of lattice structures 130. In some instances, the body 120 and the surface lattices 125 may be coupled to one another. For example, a lattice strut 135b of the body 120 may extend to a lattice juncture 135a or a lattice struts 135b of the surface lattice 125.

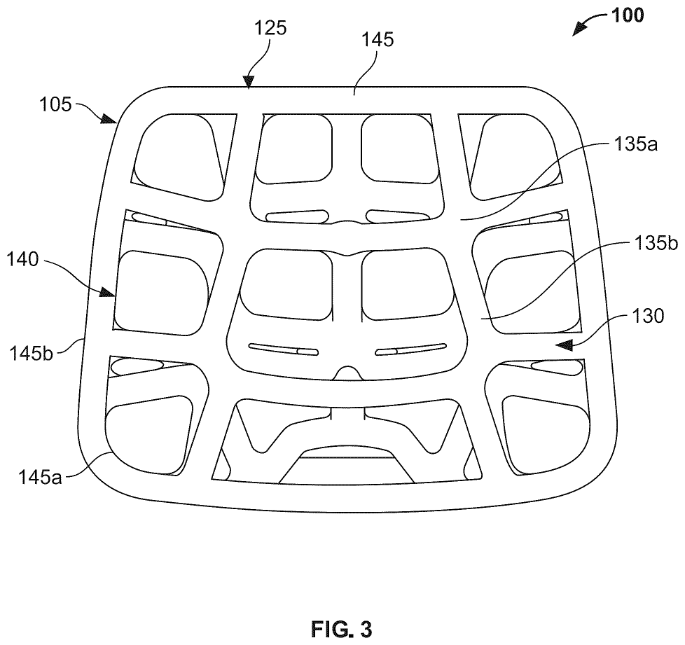

Referring to FIG. 3, a perimeter 145 of the top surface 105 may include a first edge 145a and a second edge 145b, where the first edge 145a is an inner edge and the second edge 145b is an outer edge. In various instances, the first edge 145 may be configured to bound the surface lattice 125. Further, the perimeter 145 may be substantially solid and free of lattice structures 130. In various instances, although not illustrated, the bottom surface 115 may be substantially similar to the top surface 105. That is, the bottom surface 115 may similarly include a perimeter bounding the surface lattice 125.

Advantageously, the open framework of the lattice structures 130 may provide improved mechanical properties and stability. More particularly, in certain instances, the open framework of the lattice structures 130 may be configured to impart a uniform distribution of forces across the lattice structure implant 100. In some instances, the mechanical strength and the modulus of elasticity of the lattice structure implant 100 may substantially mimic the strength, porosity, and mechanical properties of the surrounding bone of a patient. Alternatively, the lattice structure implant 100 may be relatively softer or harder. In various instances, the organic and variable lattice structures 130 may improve the endurance and adaptability of the lattice structure implant 100 while under compression and compression-shear loads. For example, the lattice structures 130 may reduce the stiffness of the lattice structure implant 100, thus allowing for the lattice structure implant 100 to bend, conform, or otherwise endure applied loads.

In various instances, the lattice structure implant 100 may be imparted with a Young's Modulus of about 5 GPa to about 20 GPa, although the Young's Modulus may be somewhat less than or even greater than these values. For example, the lattice structure implant 100 may be imparted with a Young's Modulus of at least about 5 GPa, or at least about 6 GPa, or at least about 7 GPa, or at least about 8 GPa, or at least about 9 GPa, or at least about 10 GPa, or at least about 11 GPa, or at least about 12 GPa, or at least about 13 GPa, or at least about 14 GPa, or at least about 15 GPa, or at least about 16 GPa, or at least about 17 GPa, or at least about 18 GPa, or at least about 19 GPa, or at least about 20 GPa. In some instances, the Young's Modulus may fall within a range bounded by any minimum value or maximum value as described above. In other instances, the Young's Modulus may be provided as a range that may be bounded by any minimum value and any maximum value as described above. For example, the lattice structure implant 100 may be imparted with a Young's Modulus that is about 8 GPa to about 15 GPa.

As previously described, the lattice structures 130 may include an open framework, where the open framework may include hollow areas, allowing for enhanced osteogenesis. More particularly, the relatively hollow areas may permit bone from the surrounding area to grow from and through the lattice structure implant 100. In various instances, a center of the lattice structure implant 100 may be hollow to receive and retain a bone graft. In some instances, the lattice structures 130 may be configured to impart an air to solid ratio of the lattice structure implant 100 of about 70% to about 90%, although the air to solid ratio may be less than or even greater than these values. For example, the lattice structure implant 100 may be imparted with an air to solid ratio of at least about 70%, or at least about 75%, or at least about 80%, or at least about 85%, or at least about 90%. In some instances, the air to solid ratio may fall within a range bounded by any minimum value or maximum value as described above. In other instances, the air to solid ratio may be provided as a range that may be bounded by any minimum value and any maximum value as described above. For example, the lattice structure implant 100 may be imparted with an air to solid ratio that is about 73% to about 86%.

In various instances, the lattice structure implant 100 may be configured to reduce load deflection while optimizing the strain distribution amongst the lattice structures 130. In such instances, outer edges of the lattice structure implant 100 (e.g., the first and second edges 145a, 145b and relative surrounding regions of the lattice structure implant 100) may be stiffer than an inner area (e.g., towards a center or core of the lattice structure implant 100). For example, the perimeter 145 of the top and bottom surface 105, 115 may be the stiffest regions of the lattice structure implant 100, while, towards a central and inner region of the lattice structure implant 100, the stiffness may gradually decrease. In another example, the most exterior regions of the lattice structure implant 100 may be the stiffest regions of the lattice structure implant 100, where the stiffness of the lattice structure implant 100 decreases towards a center. In some instances, the lattice structures 130 may be formed such that strain may be driven to and converge at one or more sections of the lattice structure implant 100. Further, under normal loading conditions, due to the tension and compression of the surrounding bodily environment, the strain endured by the lattice structure implant 100 may facilitate osseointegration and thereby permit fusion to occur throughout at least a portion of the lattice structure implant 100. In some instances, fusion may occur throughout the entirety of the lattice structure implant 100, due to at least the porous construction of the lattice structure implant 100.

In some instances, specific regions, areas, or portions of the lattice structure implant 100 may be altered to influence mechanical performance. For example, before, during, or after manufacturing of the lattice structure implant 100, a specific region of the lattice structure implant 100 may be selected to have a desired mechanical performance. To achieve the desired mechanical performance, components of the lattice structure implant 100, such as the lattice struts 135b, may be altered (e.g., thickness, smoothness, coarseness, and the like). One benefit of targeted alterations is that the lattice structure implant 100 may be patient specific. In some instances, the lattice structure implant 100 may have certain areas modulated to provide support for patient specific anatomy and address patient-specific needs. For example, a lattice structure implant 100 may include surface lattices 125 having thicker lattice structures 130 relative to the body 120. In this example, the lattice structure implant 100 may withstand larger loads.

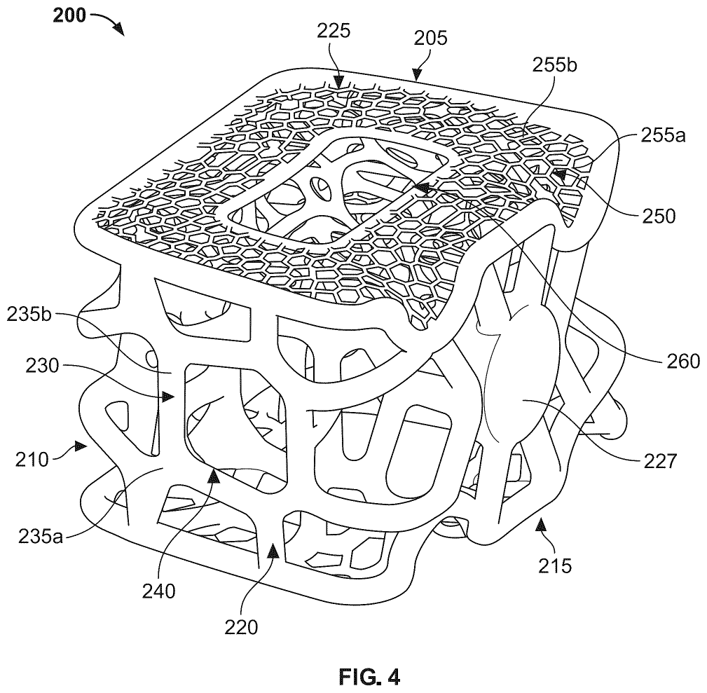

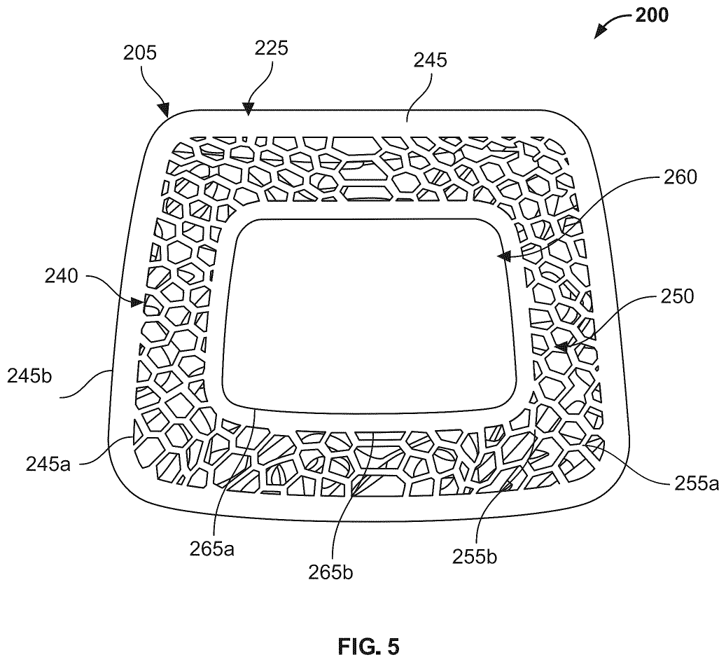

Turning to FIGS. 4 and 5, an example lattice structure implant 200 is provided. The lattice structure implant 200 may be substantially similar to the lattice structure implant 100 described with reference to FIGS. 1-3. For example, the lattice structure implant 200 may incorporate similar numbers and/or names as those corresponding elements of the lattice structure implant 100 with reference to FIGS. 1-3. Like the lattice structure implant 100, the lattice structure implant 200 may comprise a top surface 205, one or more side surfaces 210, and a bottom surface 215 that is opposite the top surface 205. Positioned about medially of one of the side surfaces 210, the lattice structure implant 200 may include a solid element 227 that is substantially similar to the solid element 127 of the lattice structure implant 100. In some instances, the solid element 227 may be configured to receive screws, rods, bars, and the like to enhance coupling of the lattice structure implant 200 to the surrounding environment upon insertion. Further, similar to the body 120 and the surface lattices lattices 125, the lattice structure implant 200 may include a body 200 and lattice surfaces 225, where the body 220 is positioned in a volume between the top surface 205, the one or more side surfaces 210, and the bottom surface 215. Moreover, the surface lattice 225 may be positioned about each the top surface 205 and the bottom surface 215. Yet further, the body 220 and the surface lattices 225 may include a plurality of lattice structure 230 that are substantially similar to the lattice structure 130. Each the lattice structures 230 may comprise one or more lattice junctures 235a connected by one or more lattice struts 235b, thus forming one or more geometric patterns 240. Additionally, the top surface 205 may be defined by a perimeter 245 having a first edge 245a and a second edge 245b, where the first and second edges 245a, 245b bound one of the surface lattices 225. In various instances, like the lattice structure implant 100, the bottom surface 215 may also include a perimeter similarly having a first edge and second edge bounding another surface lattice 225.

As illustrated in FIG. 4, the lattice structure implant 200 may include a plurality of microlattice structures 250. Whereas the body structure 220 may comprise a plurality of lattice structures 230, the surface lattices 225 may comprise a plurality of microlattice structures 250. Compared to the lattice structures 130, 230, the microlattice structures 250 may be smaller. In some instances, the microlattice structures 250 may be less dense. In other instances, the microlattice structures 250 may be scaled down relative to the lattice structures 230. For example, the microlattice structures 250 may be about 20% the density of the lattice structures 230. In other examples, the microlattice structures 250 may be less than or greater than 20% the density of the lattice structures 230. In alternative instances, the body structure 220 and the surface lattices 225 may both comprise entirely of microlattice structures 250. In yet other alternative instances, the body structure 220 and the surface lattices 225 may comprise a combination of lattice structures 230 and microlattice structures 250.

Further, the lattice structure implant 200 may also include an annular channel 260 configured to facilitate bone growth. The annular channel 260 may be an opening through the lattice structure implant 200 extending from the top surface 205 to at least a center of the lattice structure implant 200. In various instances, the annular channel 260 may linearly extend from the top surface 205 to the bottom surface 215. In various instances, the annular channel 260 may be positioned about a center of the lattice structure implant 200. As best illustrated in FIG. 5, the annular channel 260 may include four sides, forming a generally trapezoidal or rectangular shape. In alternative instances, the annular channel may be imparted with a relatively rounded periphery such that, extending from the top surface 205 to the bottom surface 215, the annular channel 260 may be relatively cylindrical. A length and a width of the annular channel 260 may be at least partially based on a size of a bone graft, patient anatomy, location of the implant, and patient needs. For example, if a bone graft is relatively block-shaped, then the periphery of the annular channel may be substantially angular such that, from the top surface 205 to the bottom surface 215, the annular channel 260 may be imparted with a substantially cubic or rectangular shape. In some instances, the annular channel may be substantially rectangular having rounded corners.

In various instances, the annular channel 260 may have a perimeter that is defined by a third edge 265a and a fourth edge 265b, where the third edge 265a is the inner most edge, defining an opening for a bone graft, and the fourth edge 265b is an outer edge, relative to third edge 265a. In some instances, a width dimension may separate the third edge 265a from the fourth edge 265b. In some instances, the annular channel 260 may comprise at least four sides. In alternative instance, the annular channel 260 may comprise less than four sides.

Continuing to reference FIG. 5, the top surface 205 may include a surface lattice 225 consisting of a plurality of microlattice structures 250. As previously described, the microlattice structures 250 may be substantially similar to the lattice structures 130, 230. That is, the microlattice structures 250 may be configured to function substantially similarly as the lattice structures 130, 230. Further, the microlattice structures 250 may be formed substantially similar as the lattice structures 130, 230.

Specifically, the surface lattices 225 may comprise a plurality of microlattice structures 250. Like the lattice structures 130, 230, the microlattice structures 250 may include a framework consisting of a series of intersecting elements, thus forming a grid-like or network pattern. The microlattice structures 250 may comprise a plurality of microlattice junctures 255a connected by a plurality of microlattice struts 255b. Generally, microlattice junctures 255a may be points or nodes at which one or more microlattice struts 255b may converge. For example, two microlattice struts 255b may converge at a single microlattice juncture 255a. In another example, three microlattice struts 255b may converge at a single microlattice juncture 255a. In yet another example, four microlattice struts 255b may converge at a single microlattice juncture 255a.

To form the microlattice structure 250 in a surface lattice 225, a plurality of microlattice junctures 255a may be dispersed throughout an area of the top surface 205. Each of the microlattice junctures 255a may be connected by one or more microlattice struts 255b. The interconnections between the microlattice struts 255b at each of the microlattice junctures 255a, may substantially formed the overall grid-like or network pattern of the microlattice structures 250.

As microlattice struts 255b connect to one more microlattice junctures 255a, a geometric pattern 240 of a microlattice structure 250 may be formed. The geometric pattern 240 may be at least partially based on the surrounding microlattice junctures 255a and microlattice struts 255b. In particular, in some instances, the positioning, amount, and distance between the microlattice junctures 255a and the curvature or linearity of the microlattice struts 255b connecting each microlattice junctures 255a may at least partially influence the geometric pattern 240. For example, four microlattice junctures 255a may be connected via four substantially linear microlattice struts 255b, thus forming four convergence areas. Accordingly, the microlattice structure 250 formed from the four microlattice junctures 255a and the four microlattice struts 255b may be imparted with a geometric pattern 240 that is substantially rectangular. In some instances, a microlattice structure 250 may be imparted with a geometric pattern 240 that is substantially trapezoidal or rectilinear. In another example, the microlattice structure 250 may be imparted with a geometric shape 240 that is substantially rounded, circular, and the like. For example, a microlattice strut 255b connecting microlattice junctures 255a may have a substantially arcuate, bowed, or curved shape. As such, the microlattice strut 255b may provide a rounded aspect to the formed geometric pattern 270. In yet another example, the microlattice structure 250 may be imparted with a geometric pattern 270 that is substantially abstract. In some instances, a microlattice strut 255b connecting two microlattice junctures 255a may be imparted with a substantially hour-glass shape. In other instances, a microlattice strut 255b connecting two lattice microlattice junctures 255a may be imparted with a substantially sinusoidal shape, such that the microlattice strut 255b forms peaks and troughs between the two microlattice junctures 255a. In various instances, the microlattice structure 250 includes various geometric patterns 270.

The surface lattice 225 may comprise a plurality of microlattice structures 250. In some instances, the plurality of microlattice structures 250 may be substantially identical to each other. For example, a first microlattice structure 250 may be formed, and a second microlattice structure 250 may be formed by substantially duplicating and translating the positioning, shape, and elements of the first microlattice structure 250 (e.g., the microlattice junctures and struts 255a, 255b), thus forming a substantially identical second microlattice structure 250. In other instances, a first microlattice structure 250 may be formed, and a second microlattice structure 250 may be formed by reflecting, rotating, or translating elements of the first microlattice structure 250 (e.g., the microlattice junctures and struts 255a, 255b) over the X-axis, the Y-axis, and/or the Z-axis. In such instances, the second microlattice structure 250 may be substantially similar to the first microlattice structure 250. Generally, the surface lattice 225 may comprise a plurality of microlattice structures 250, where each microlattice structure 250 of the plurality of microlattice structures 250 includes repeated elements that are either substantially identical or substantially similar (e.g., mirrored over the X-axis, Y-axis, or Z-axis). Alternatively, the microlattice structures 250 may be substantially complex, consisting of non-repetitive or irregular configurations of the microlattice junctures and struts 255a, 255b

In various instances, the microlattice structures 250 may couple to the lattice structures 230, such that the body 220 and the surface lattices 225 are connected. In such instances, about the top surface 205, the lattice struts 235b may gradually become thinner until achieving a desired thickness and density of a microlattice struts 255b. In various instances the body 120 and the surface lattices 125 may be integrally formed.

Although not illustrated, the features of the top surface 205 and the corresponding surface lattice 225 may be similarly implemented to the bottom surface 215. That is, the bottom surface 215 may be substantially similar to the top surface 205 and may include substantially similar microlattice structures 250.

As illustrated in FIG. 5, the surface lattice 225, comprising a plurality of microlattice structures 250, may be positioned between a first edge 245a and a fourth edge 265b. In some instances, the lattice structure implant 200 may not include the fourth edge 265b. In such instances, the surface lattice 225 may be positioned between the first edge 245a and a third edge 265a. In other instances, the lattice structure implant 200 may not include the first edge 245a. In such instances, the surface lattice 225 may be positioned between a second edge 245b and the fourth edge 265b. In yet other instances, the lattice structure implant 200 may not include the first edge 245a and the fourth edge 265b. In such instances, the surface lattice 225 may be positioned between the second edge 245b and the third edge 265a. In some instances, the microlattice structures 250 and/or lattice structures 230 may be bounded by any of the four or more edges. In alternative instances, the microlattice structures 250 and/or lattice structures 230 may not be bounded by any one or more edges. In yet other alternative embodiments, the lattice structure implant 200 may not include any edges.

The distance between the third edge 265a and the fourth edge 265b may be of substantially similar thickness as the microlattice struts 235a. In other instances, the distance between the third edge 265a and the fourth edge 265b may be of lesser or greater thickness than the microlattice struts 255a. In some instances, the first edge 245a and the second edge 245b may be of substantially similar thickness as the lattice struts 235b. In other instances, the distance between the first edge 245a and second edge 245b may be of lesser or greater thickness than the lattice struts 235b. In other instances, the distance between the first edge 245a and second edge 245b may be substantially similar to the distance between the third edge 265a and fourth edge 265b. In other instances, the distance between the first edge 245a and second edge 245b may be different than the distance between the third edge 265a and fourth edge 265b.

Moreover, the lattice structure implant 200 may be imparted with a microlattice offset. The microlattice offset may be a gap between the surface lattice 225 and the body 220. The microlattice offset may be controlled by a user-defined variable. In various instances, the microlattice offset may remain constant across a line of the lattice structure implant 200.

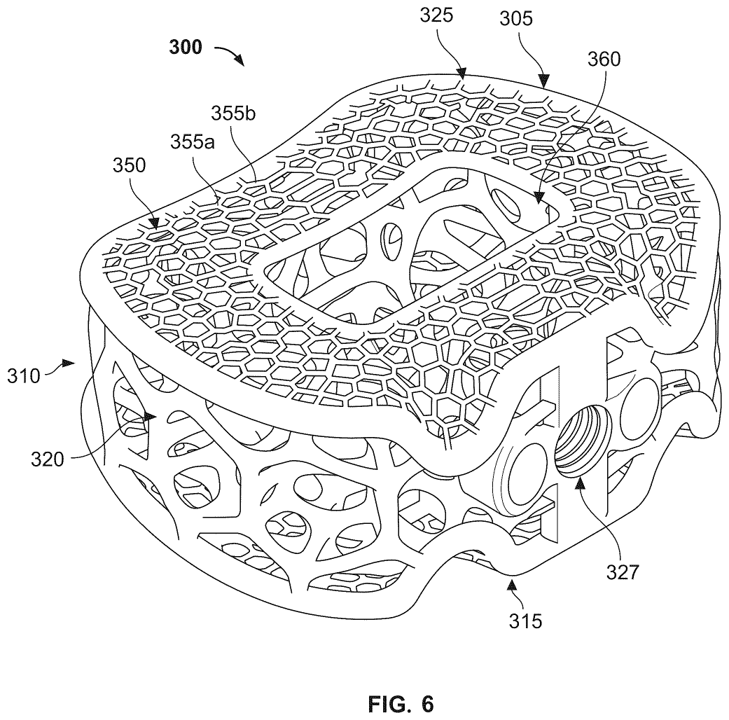

Turning to FIG. 6, another example of a lattice structure implant 300 is provided. The lattice structure implant 300 may be substantially similar to the lattice structure implant 100, 200 described with reference to FIGS. 1-5. In some instances, the lattice structure implant 300 may be substantially similar to the lattice structure implant 200 described with reference to FIGS. 4 and 5. For example, the lattice structure implant 300 may incorporate similar numbers and/or names as those corresponding elements of the lattice structure implant 200 with reference to FIGS. 4 and 5. Like the lattice structure implant 200, the lattice structure implant 300 may include a top surface 305, one or more side surface 310, and bottom surface 315 opposite the top surface 305. Further, like the lattice structure implant 200, the lattice structure implant 300 may include a body 320 including a plurality of lattice structures 330, and surface latices 325 including a plurality of microlattice structures 350. Generally, the lattice structure implant 300 may be formed similarly as lattice structure implants 100, 200 described with reference to FIGS. 1-5. Further, the lattice structure implant 300 may include one or more bores 327 configured to engage with other hardware such as screws, inserts, and rods, amongst others. In some instances, the lattice structure implant 300 may be utilized for surgical techniques such as ALIF.

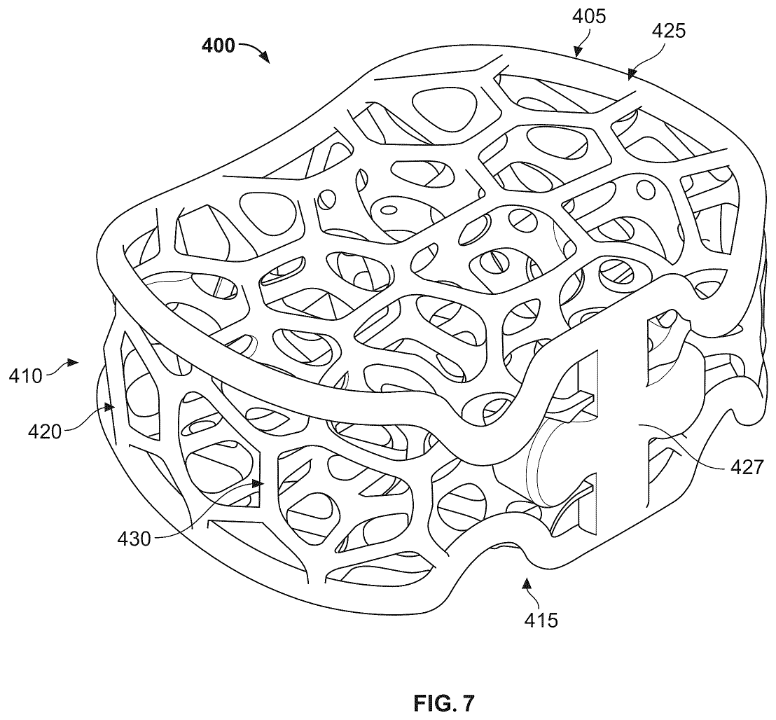

Turning to FIG. 7, yet another example of a lattice structure implant 400 is provided. The lattice structure implant 400 may be substantially similar to the lattice structure implant 100, 200 described with reference to FIGS. 1-6. In some instances, the lattice structure implant 400 may be substantially similar to the lattice structure implant 100 described with reference to FIGS. 1-3. For example, the lattice structure implant 400 may incorporate similar numbers and/or names as those corresponding elements of the lattice structure implant 100 with reference to FIGS. 1-3. Like the lattice structure implant 100, the lattice structure implant 400 may include a top surface 405, one or more side surface 410, and bottom surface 415 opposite the top surface 405. Further, like the lattice structure implant 100, the lattice structure implant 400 may include a body 420 and surface lattices 425 including a plurality of lattice structures 430. Generally, the lattice structure implant 400 may be formed similarly as lattice structure implants 100, 200, 300 described with reference to FIGS. 1-6. In some instances, like the lattices structure implant 300, the lattice structure implant 400 may be utilized for ALIF. In various instances, a solid element 427 of the lattice structure implant 400 may be substantially linear.

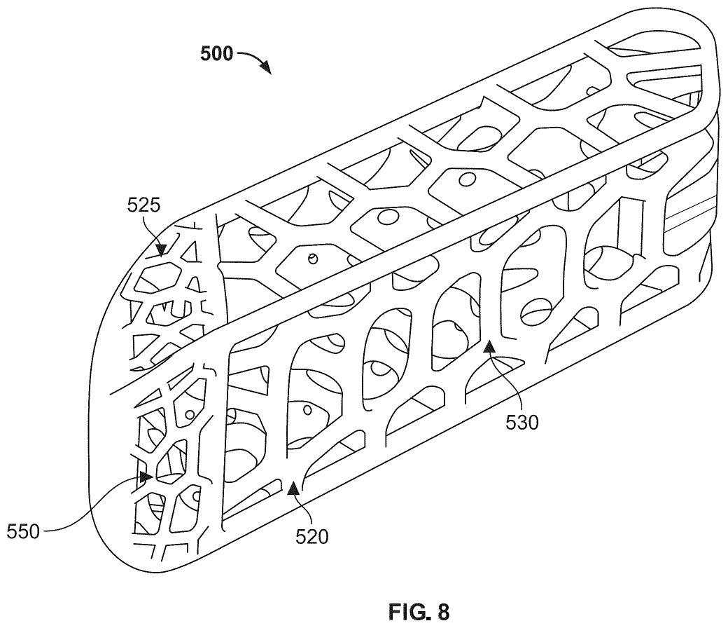

Turning to FIG. 8, an example of a lattice structure implant 500 is provided. In various instances, the lattice structure implant 500 may be utilized for surgical techniques such as TLIF. The lattice structure implant 500 may be substantially similar to the lattice structure implant 100, 200, 300, 400 described with reference to FIGS. 1-7. In some instances, lattice structure implant 500 may be substantially similar to the lattice structure implant 200, 300, described with reference to FIGS. 4-6. Like the lattice structure implant 200, 300 the lattice structure implant 500 may include a body 520 and surface lattices 525. The body 520 may include a plurality of lattice structures 530, and the surface lattices 525 may include a plurality of microlattice structure 550. In various instances, the lattice structure implant 500 may include the plurality of microlattice structures 550 on one or more lateral sides of the lattice structure implant 500. In such instances, the lattice structures 530 and the microlattice structures 550 may substantially occupy a volume of the lattice structure implant 500. Further, the lattice structure implant 500 may be imparted with a shape that is substantially similar to rectangular prism (i.e., the lattice structure implant 500 may be longer than wider).

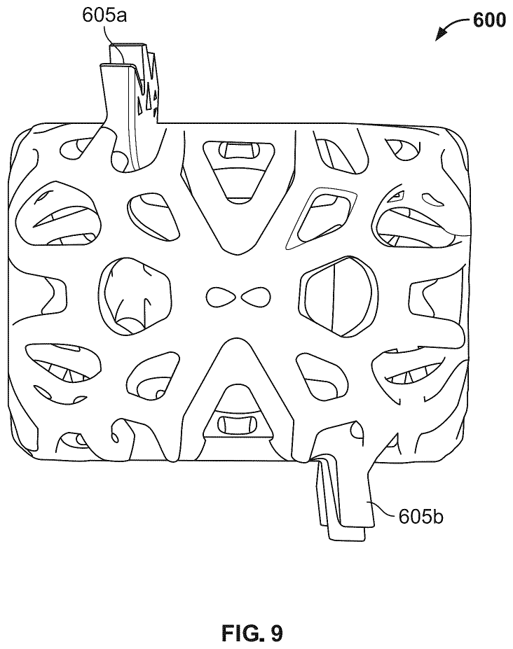

Turning to FIGS. 9, an example lattice structure implant 600 is provided. The lattice structure 600 may be substantially similar to lattice structure 100, 200, 300, 400, 500, described with reference to FIGS. 1-8, and may further include one or more keels 605. The keels 605 may be configured to facilitate bone fixation between the lattice structure implant 600 and the surrounding environment. In other words, the keels 605 may function substantially similar to an anchor, where the keels 605 may couple the lattice structure implant 600 to surrounding bones. The keels 605 may be coupled to any side of the lattice structure implant 600. As illustrated in FIGS. 9, a first pair of keels 605a may be positioned about a first side of the lattice structure implant 600, and a second pair of keels 605b may be positioned about an opposite side of the lattice structure implant 600. In some instances, the first pair of keels 605a may be positioned about a top surface and a second pair of keels 605b may be positioned about an opposing, bottom surface. Generally, the keels 605 may have a spiked, triangular form, with a point of the triangle facing exteriorly from the center of the lattice structure implant 600 such that the point may engage with at least one of the surrounding rigid bones. In other instances, keels 605 may be of any other angular shape such that a point of the shape is configured to couple to a surrounding rigid bony feature of a patient's body.

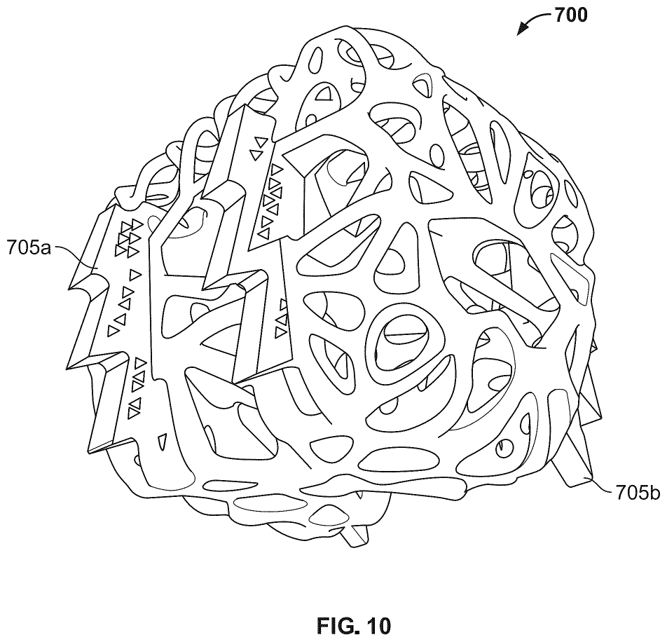

Turning to FIG. 10, an example lattice structure implant 700 is provided. The lattice structure implant 700 may be substantially similar to the lattice structure implant 600, described with reference to FIG. 9, in that the lattice structure implant 700 may include one or more pairs of keels 705. Like the lattice structure implant, the lattice structure implant 700 may include a first pair of keels 705a and a second pair of keels 705, both pair of keels 705 (i.e., the first pair of keels 705a and the second pair of keels 705b) may be configured to anchor or secure the lattice structure implant 700 to the surrounding environment.

In various instances, a method for manufacturing the lattice structure implant 100, 200, 300, 400, 500, 600, 700, described with reference to FIGS. 1-10, may include the execution of a specific algorithm. In particular, the algorithm provides an outcome (e.g., the lattice structure implant 100, 200, 300, 400, 500, 600, 700) that otherwise could not be provided, achieved, accomplished, or done without the use of said specific algorithm. In some instances, a remote device or a computation component that is configured (e.g., via one or more processors) to execute the algorithm. Generally, the method of manufacturing may include a step directed to executing an algorithm configured to generate, develop, and form the entirety of the lattice structure implant 100, 200, 300, 400, 500, 600, 700. In some instances, the lattice structure implant 100, 200, 300, 400, 500, 600, 700 may be manufactured by 3D printing using direct metal laser sintering (DMLS). In various instances, the lattice structure implant 100, 200, 300, 400, 500 600, 700 may be modified via Boolean operations such as Boolean union, Boolean subtract, and Boolean Intersect may be used.

Moreover, the algorithm may include three phases. A first phase may be directed to forming the body 120, 220, 320, 420, 520. A second phase may be directed to forming the surface lattices 125, 225, 325, 425, 525. A third phase may be directed to forming, if applicable, at least one or more additional solid elements 127, 227, 327, 427 or keels 600 and combining the body 120, 220, 320, 420, 520 and surface lattices 125, 225, 325, 425, 525 to form the lattice structure implant 100, 200, 300, 400, 500, 600. It is to be understood that, although not illustrated, the lattice structure implants 100, 200, 300, 400, 500, 600, 700 described herein may all include substantially similar components such as a body and one or more surface lattices, the body and the one or more surface lattices each comprising a plurality of lattices structures.

The first phase of the algorithm may comprise multiple steps. First, the algorithm may generate Voronoi lattices that may be at least partially based on point mapping and expanding points therefrom. In some instances, the method may include a step of developing an initial framework or foundation of the desired points for the lattice structure implant 100, 200, 300, 400, 500, 600, 700. In various instances, the first part of the algorithm may be directed to forming one or more lattice structures 130, 230, 330, 430, 530 for at least about one-fourth of a defined initial or final volume of the lattice structure implant 100, 200, 300, 400, 500. The initial framework may include defining and/or identifying the width, shape, volume limits (e.g., a desired final volume of the implant), depth, implant posterior height, and lordotic angle.

As illustrated in FIG, 11, another step may include generating points (i.e., a point map 800) within about a quarter segment (i.e., a fourth) of the lattice structure implant 100. The points may be generated according to one or more input parameters that may be entered by a user via the remote device or the computational component. The parameters may relate to point density (e.g., a point per volume), tolerance and limit values, points in a volume, infill volume, spacing between points, surface structure of the infill volume, patient-specific values, additional user-desired input, amongst others. Using the input parameters, the point map 800 may be generated. In some instances, the points may be bounded by the volume limits previously inputted by the user. By bounding the points to be within volume limits, the lattice structures 130, 230, 330, 430, 530 may maintain a closed configuration. That is, the lattice structures 130, 230, 330, 430, 530 may have substantially no opening between the points (e.g., lattice juncture 135a), thus maintain connection between the lattice structures 130, 230, 330, 430, 530. Therefore, the desired final shape of the lattice structure implant 100 may be maintained.

In some instances, the positioning of the one or more points may be described with respect to an abscissa axis (“X-axis”), an ordinate axis (“Y-axis”), and an applicate axis (“Z-axis”) of the lattice structure implant 100, 200, 300, 400, 500, 600, 700. For example, a first point may be positioned on an anterior face of the lattice structure implant 100 (such as the top surface 105) at about a 45° angle with respect to the X-and/or Y-axes. In some instances, a point may be made at either a center of the anterior face, where the “center” may be a point of intersection between the X-axis, Y-axis, and Z-axis. In various instances, the point of intersection between the X-axis, Y-axis, and Z-axis may be the middle of the lattice structure implant 100 with respect to length, width, and depth. After the first point is generated on the point map 800, one or more points may be generated on the same surface outwardly from the first point following volume limits and tolerance parameters inputted by a user. In some instances, the maximum allowable and/or desired quantity of points may be based on a predetermined quantity calculated by the algorithm. Generally, points may be made according to the parameters inputted by a user into the algorithm and according to a patient's individual needs.

The point map 800 may be configured to generate the body 120, 220, 320, 420, 520 by creating points 805 and forming a wire graph consisting of one or more spheres 810 that may expand from each point 805 on the point map. A line may be formed at each point of intersection between each expanded sphere 810 (i.e., a tangent line may be formed between each expanded sphere). In various instances, the expanded spheres 810 may be similarly bounded to be within the volume limits. For example, the volume limits may be set to represent the edges of the final lattice structure implant 100, 200, 300, 400, 500, 600, 700. Continuing this example, by maintaining points 805 within the boundaries, the integrity and desired shape of the final lattice structure implant 100, 200, 300, 400, 500 may be maintained. At each point 805 on the point generated map, the surrounding volume may spherically expand therefrom until reaching the volume limit. Generally, the sphere 810 may be an initial outline of the lattice structures 130, 230, 330, 430, 530.

One benefit amongst others of generating a point map 800 and wire graphing is that the lattice structure implant 100, 200, 300, 400, 500, 600, 700 may formed at least partially independent of parameters. For example, upon generating the point map 800 and wire graphing, changing parameter values may minimally impact the overall design of the lattice structure implant 100. In alternative instances, changing a number of parameter values, or changing at least one integral parameter value, may impact the design of the lattice structure implant 100, 200, 300, 400, 500, 600, 700.

In various instances, the generation of the lattice structures 130, 230, 330, 430, 530 may be at least partially based on patient-specific parameters, creating a robust and unique lattice structure implant 100, 200, 300, 400, 500, 600, 700 for each individual patient. In some instances, the lattice structures 130, 230, 330, 430, 530 may be of differing shapes and sizes from each other within the lattice structure implant 100, 200, 300, 400, 500, 600, 700. In alternative embodiments, the lattice structures 130, 230, 330, 430, 530 may be of uniform shapes and sizes.

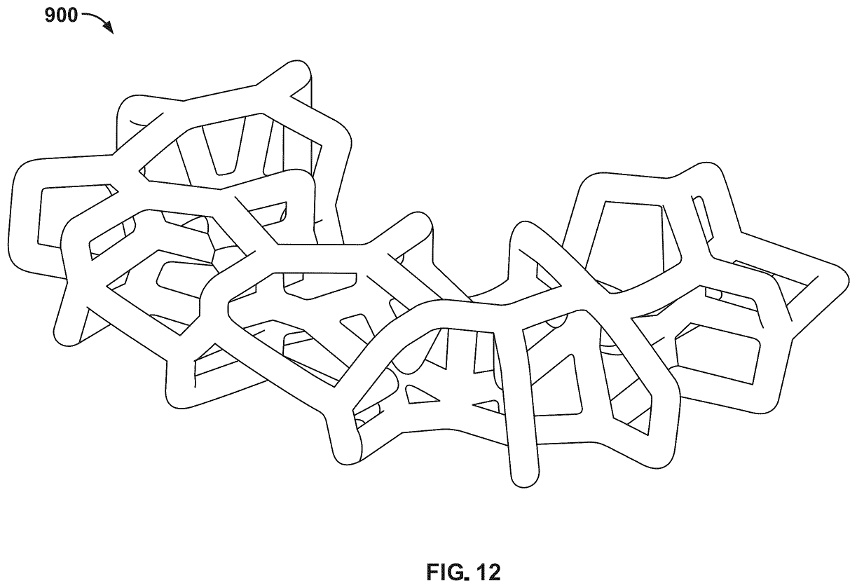

Turning to FIG. 12, an example of a quarter segment 900 of a body 120, 220, 320, 420, 520 is provided. As illustrated in FIG. 12, the method of manufacturing may include a step of thickening the quarter segment 900. In other words, the method of manufacturing may include a step directed to giving weight to the lattice structures 130, 230, 330, 430, 530. In some instances, the quarter segment 900 may be symmetrical with respect to the horizontal and vertical planes.

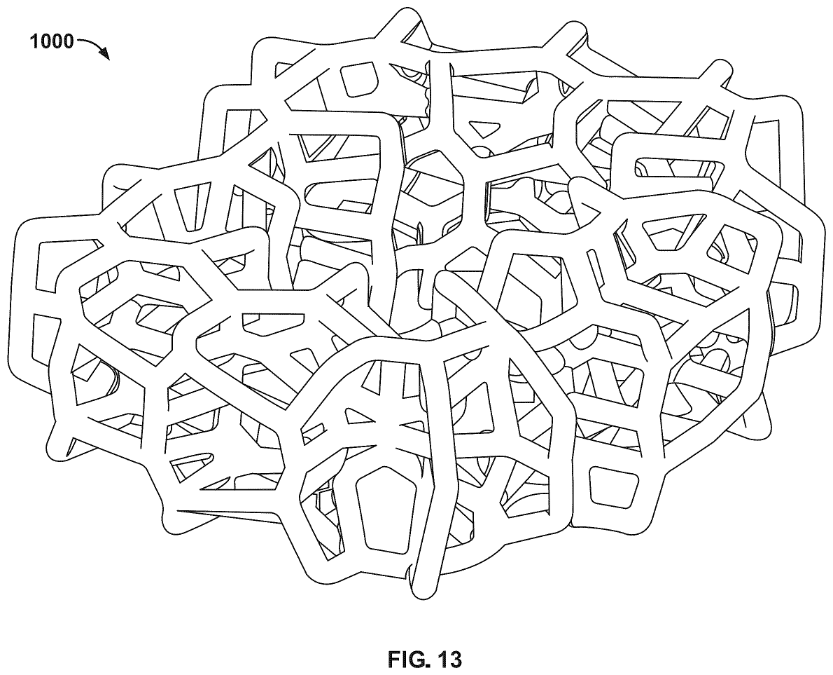

Turning to FIG. 13, after creating the first thickened quarter segment 900, the quarter segment 900 may be mirrored over the horizontal and vertical planes to form the body lattices 1000. In various instances, the body lattices 1000 may be foundational to the body 120, 220, 320, 420, 520. Moreover, due to the mirroring of the quarter segment 900, the entire lattice structure implant 100, 200, 300, 400, 500, 600, 700 may be symmetrical with respect to the horizontal and vertical axes and planes. In other words, in various instances, the lattice structure implant 100, 200, 300, 400, 500 may be symmetric about two planes. Benefits of the symmetry may include (1) a substantially even distribution of forces throughout the lattice structure implant 100, 200, 300, 400, 500, 600, 700, regardless of the orientation of the lattice structure implant 100, 200, 300, 400, 500 in a patient's body, and (2) equal load distribution in at least the medial-lateral direction on both halves of the lattice structure implant 100, 200, 300, 400, 500, 600, 700.

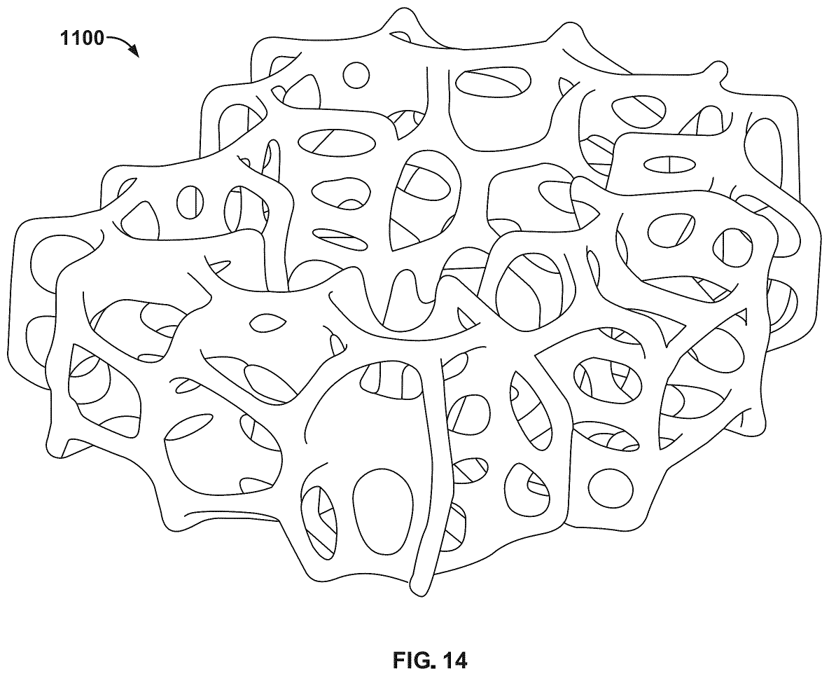

The algorithm may further include a smoothing step. The smoothing step may be directed to creating a variable thickness, resulting in a final body lattice 1100, as illustrated in FIG. 14. In various instances, the final body lattice 1100 may be the body 120, 220, 320, 420, 520. In some instances, the user may input variables (i.e., body lattice variables) such as the smoothing factor, offset, point density, and shape of space. In such instances, the final body lattice 1100 may be selectively adjusted to accommodate specific patients. For example, a thickness may be identified based on the inputted body lattice variables. In various instances, the thickness may be about 1 mm to about 2 mm, although the thickness may be somewhat less than or even greater than these values. For example, the thickness may be at least about 1 mm, or at least about 1.1 mm, or at least about 1.2 mm, or at least about 1.3 mm, or at least about 1.4 mm, or at least about 1.5 mm, or at least about 1.6 mm, or at least about 1.7 mm, or at least about 1.8 mm, or at least about 1.9 mm, or at least about 2 mm. In some instances, the thickness may fall within a range bounded by any minimum value or maximum value as described above. In other instances, the thickness may be provided as a range that may be bounded by any minimum value and any maximum value as described above.

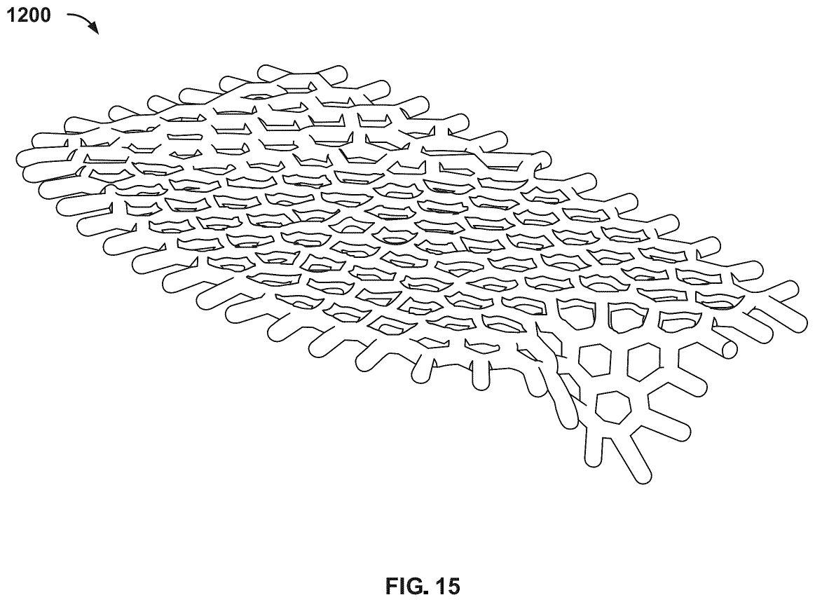

Following the formation of the final body lattice 1100, or simultaneously during the formation of the final body lattice 1100, the surface lattices 125, 225, 325, 425, 525 may be formed. Similar to the final body lattice 1100, at least a quarter portion of the one or more surface lattices 1200 may be made first. Forming the surface lattices 125, 225, 325, 425, 525 may include the step of identifying a top face. In some instances, the top face may be the face that interacts with vertebral body endplates. In some instances, the top face may become the top surface 105, 205, 305, 405 in the lattice structure implant 100, 200, 300, 400. Another step may include meshing, isolating, and downwardly translating the top face from its original position. In some instances, the top face may be translated downwards an amount that is at least about half of the thickness of the final body lattice 1100. Benefits of translating the top face downwards may include ensuring that the one or more surface lattices 1200 stay within the original bounds of the lattice structure implant 100. After a quarter potion of the surface lattice is formed, like the body lattice 1000, the quarter portion may be mirrored to form the surface lattice 1200, as best illustrated in FIG. 15.

Alternatively, but without substantially deviating from the methods and processes for manufacturing a lattice structure implant 100 described herein, rather than developing a quarter segment 900, a half segment may be developed (not illustrated). For example, the generation of the point map 800 and wire graphing may be within a half segment. Continuing this example, body lattices 1000 may be thickened for the half segment. Further, the half segment may be mirrored or substantially replicated over a single plane (e.g., a horizontal plane or a vertical plane). In some instance, when the half segment is reflected over a horizontal plane, a top and a bottom of the lattice structure implant 100, 200, 300, 400, 500, 600, 700 may be symmetrical. In other instances, when the half portion is reflected over a vertical plane, a left side and a right side of the lattice structure implant 100, 200, 300, 400, 500, 600, 700 may be symmetrical. In various instances, lattice structure implants 100, 200, 300, 400, 500, 600, 700 that are directed to supporting TLIF may be manufactured by developing a half segment rather than a quarter segment. As such, the lattice structure implants 100, 200, 300, 400, 500, 600, 700 directed to supporting TLIF may only have single-plane symmetry. In such instances, the symmetry may still support a substantially even distribution of forces throughout the implant and equal load distribution in a medical or lateral direction.

In some instances, the smoothing step may be accomplished by a Gaussian smoothing algorithm. In such instances, the inputs may include a grid size of about 0.125 mm to about 0.4 mm. The grid size may be used or indicative of the spacing between sample points used in the algorithm for manufacturing the lattice structure implant 100, 200, 300, 400, 500, 600, 700 or how many times the algorithm is executed to generate a final smoothened body. In some instances, a larger grid size may result in a relatively smoother body, as there is greater space between sample points. For larger grid sizes, the geometry of the resulting smoothened body may deviate from the original structure of the lattice structure implant 100, 200, 300, 400, 500, 600, 700 more than a body that was smoothed following a smaller grid size. A larger grid size may also impact the lattice struts 135b, 235b. For example, a larger grid size may cause the lattice struts 135b, 235b to taper such that the lattice struts 135b, 235b have an hourglass shape.

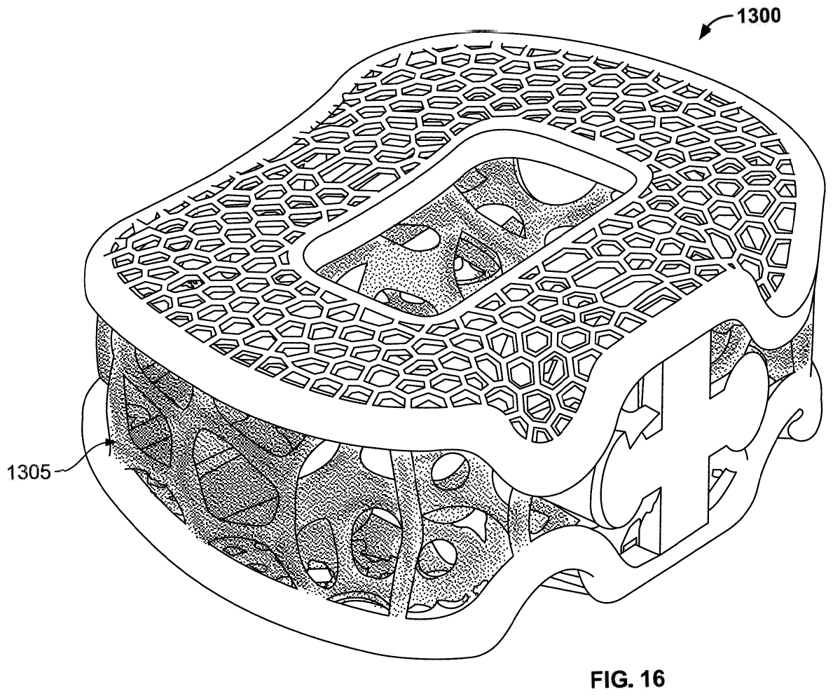

Turning to FIG. 16, an example lattice structure implant 1300 is provided. The lattice structure implant 1600 may be substantially similar to the lattice structure implant 100, 200, 300, 400, 500, 600, 700 described with reference to FIGS. 1-10. Further, the lattice structure implant 1300 may have a surface roughness 1305 about one or more sections of the lattice structure implant 1300.

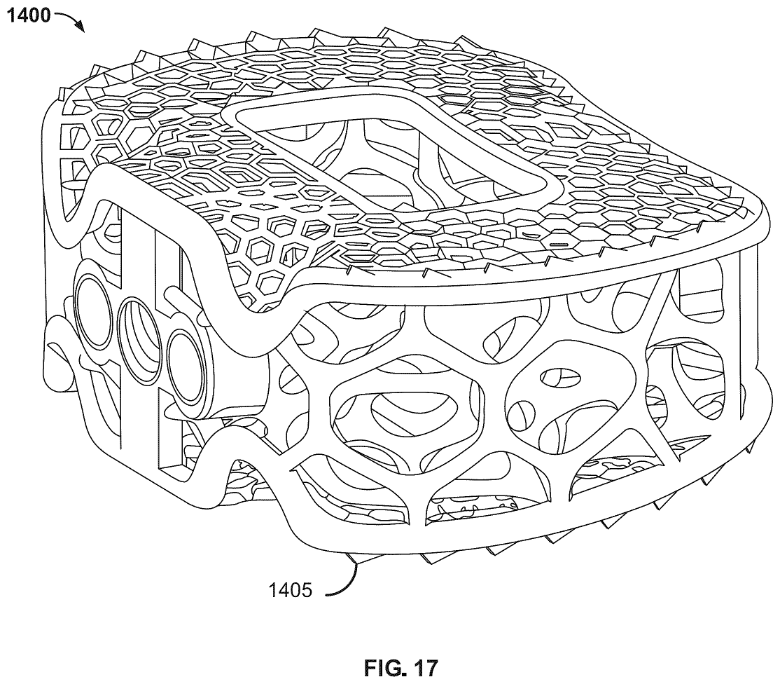

Turning to FIG. 17, an example of a lattice structure implant 1400 is provided. The lattice structure implant 1400 may be substantially similar to the lattice structure implant 100, 200, 300, 400, 500, 600, 700, 1300 described with reference to FIGS. 1-10 and 16. Further, the lattice structure implant 1400 may include serrations 1405. The serrations 1405 may be configured to secure the placement of the lattice structure implant 1400 such that lattice structure implant 1400 is resistant to expulsion or migration.

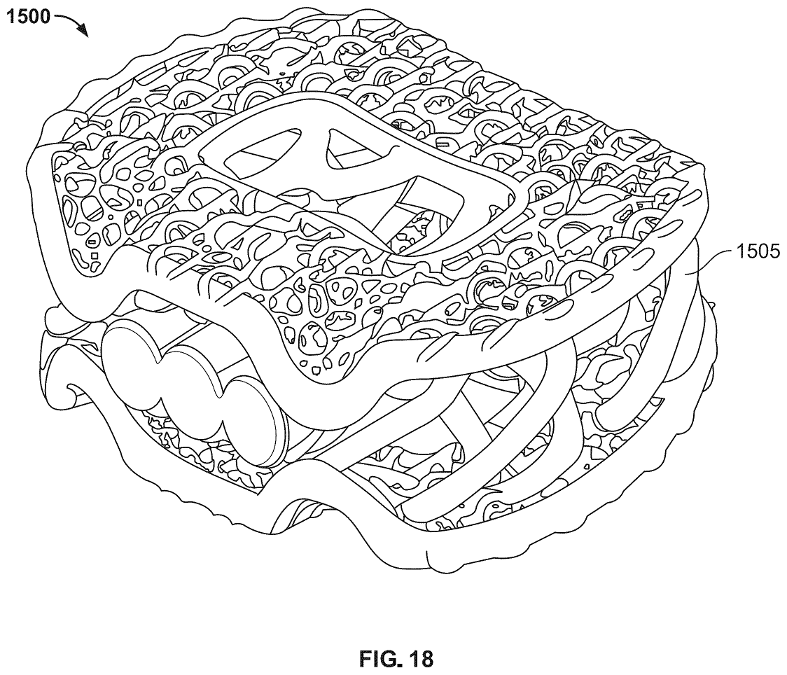

Turning to FIG. 18, another example of a lattice structure implant 1500 is provided. The lattice structure implant 1500 may be similar to lattice structure implants 100, 200, 300, 400, 500, 600, 1300, 1400 described with reference to FIGS. 1-10, 16, and 17. Further, the lattice structure implant 1500 may comprise a torus structure 1505. Rather than lattice structures, the lattice structure implant 1500 may include a torus structure 1505 that may extend through a volume of the lattice structure implant 1500.

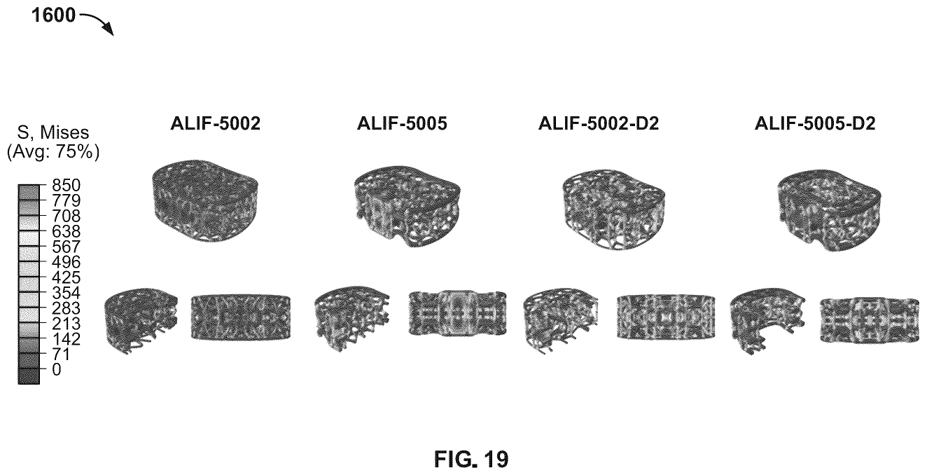

In various instances, stress may be uniformly distributed across the lattice structure implant 100, 200, 300, 400, 500, 600, 700, 1300, 1400, 1500. In some instances, the lattice structure implant 100, 200, 300, 400, 500, 600, 700, 1300, 1400, 1500 may be configured to concentrate stress to one or more designated areas and regions. In some instances, the lattice structure implant 100, 200, 300, 400, 500, 600, 700, 1300, 1400, 1500 may be configured to include one or more monomers that mimic the shape and formation of tissue cells. In other instances, the lattice structure implant 100, 200, 300, 400, 500, 600, 700, 1300, 1400, 1500 may mimic the elasticity of bony, thus reducing the possibility of stress fractures throughout the device. In such instances, the lattice structure implant 100, 200, 300, 400, 500, 600, 700, 1300, 1400, 1500 may be substantially smooth. Referring to FIG. 19, a stress diagram 1600 of various instances of the lattice structure implant 100, 200, 300, 400, 500, 600, 700, 1300, 1400, 1500 is provided. The stress diagram 1600 illustrates the distribution of stress under static compression with a load of about 22 kN, although the lattice structure implant 100100, 200, 300, 400, 500, 600, 700, 1300, 1400, 1500 may be configured to endure a load that is greater than or less than about 22 kN.

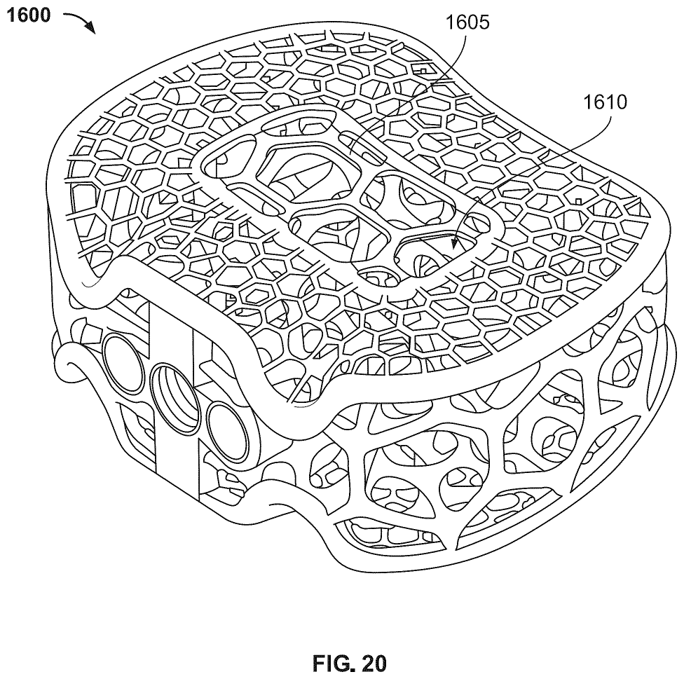

Turning to FIG. 20, yet another example of a lattice structure implant 1600 is provided. The lattice structure implant 1600 may similar to lattice structure implant 100, 200, 300, 400, 500, 600, 700, 1300, 1400, 1500 described with reference to FIGS. 1-10, 16, 17, and 18. Further, the lattice structure implant 1600 may include an annular lattice covering 1605. The annular lattice covering 1605 may be configured to extend across an annular channel 1610. In some instances, the annular lattice covering 1605 may only superficially cover an opening to the annular channel 1610. In other instances, the annular channel covering 1605 may cover an opening to the annular channel 1610 about both a top and bottom surface.

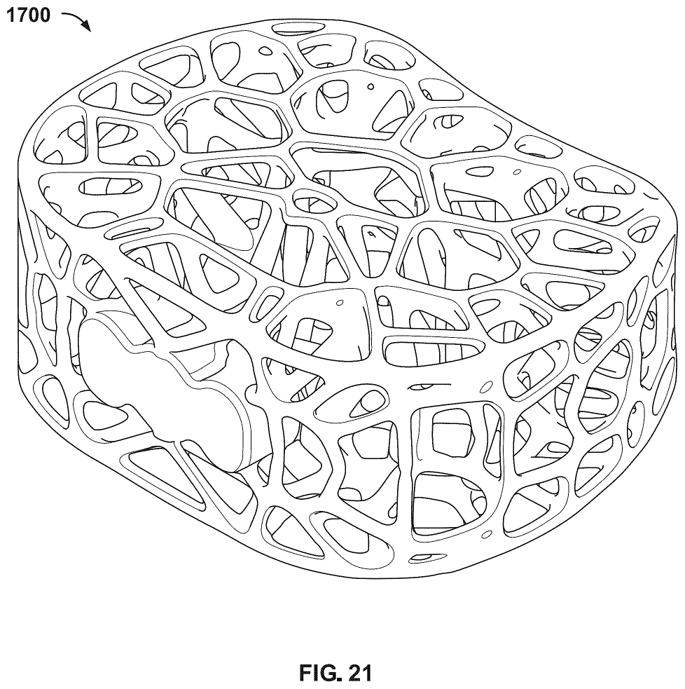

Turning to FIG. 21, a lattice structure implant 1700 is provided. The lattice structure implant 1700 may comprise substantially similar elements as the lattice structure 100, 200, 300, 400, 500, 600, 700, 1300, 1400, 1500, 1600. Further, the lattice structure implant 1700 may be formed based on location. In some instances, generating the lattice structure implant 1700 may include substantially similar steps from the methods and processes for manufacturing previously described. However, while developing points on the map, the points may be generated according to one or more additional parameters relating to location and/or distance rather than density. For example, the points for a lattice structure implant 1700 may be generated based upon a location within a certain volume which may result in the lattice struts and junctures being generated with a substantial dependence upon the location within the volume.

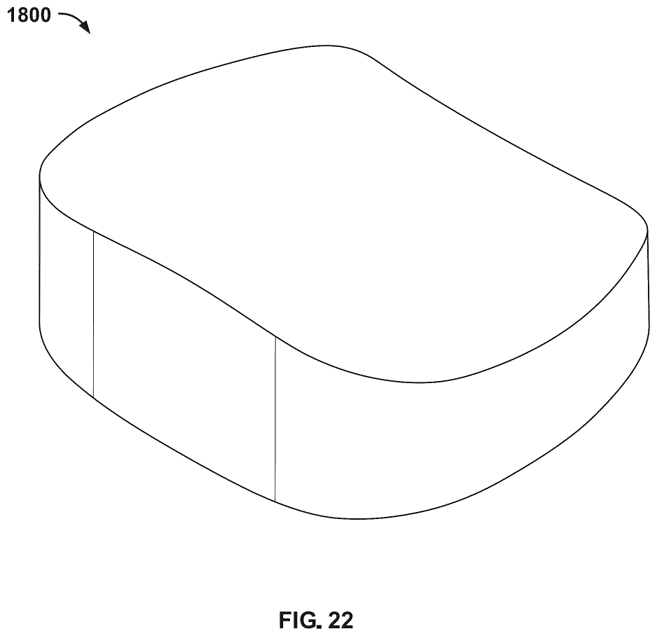

To support location and distance-based lattice generation, a body 1800 representing an offset volume may be used. As best illustrated in FIG. 22, the body 1800 may have an angular shape, a rounded shape, or a combination of angular and rounded shapes. In various instances, the body 1800 may have a tapered outer surface. In some instances, the body 1800 may be customized for each implant. During the first phase of the algorithm, the body 1800 may occupy a space or “field” such that lattice generation may be controlled and/or influenced by said field. For example, the distance between the ends of the body 1800 may determine or influence the distance between the points during lattice generation. That is, the body 1800 may substantially influence lattice generation to be dependent upon volume and distance rather than density. The points may be generated throughout the first one-fourth portion based on a minimum distance from other points, and, by basing point positions on the point map on distance, the resulting lattice density may be controlled throughout the entire volume of the lattice structure implant 1700.

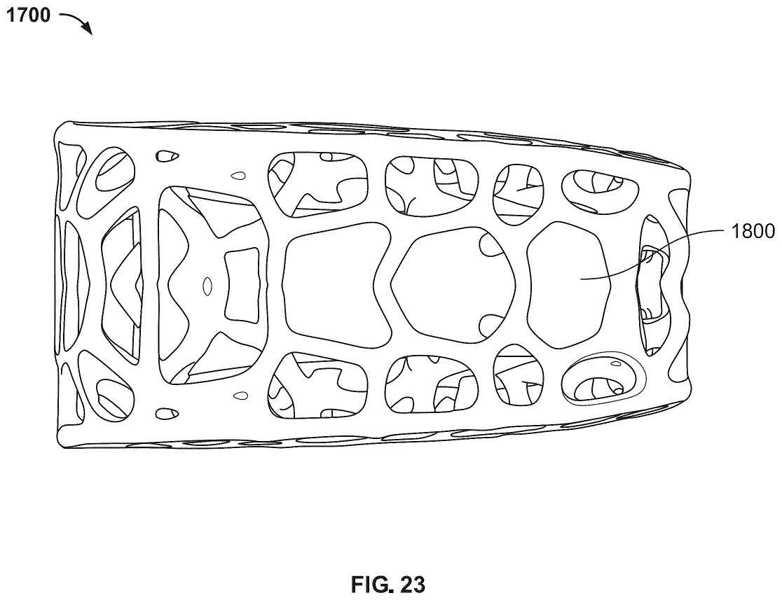

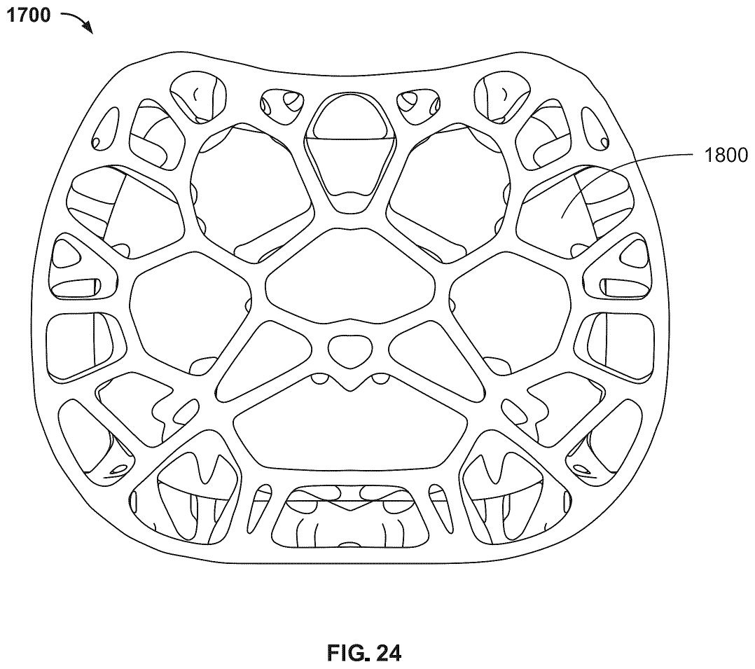

Referring to FIGS. 23 and 24, a configuration of the body 1800 within the lattice structure implant 1700 is provided. In some instances, a distance between the body 1800 and the edges of the lattice structure implant 1700 may be constant. Alternatively, the distance may be greater in some areas relative to others.

Many embodiments of an implant have been discussed in this disclosure. It is to be understood that these embodiments and examples are nonlimiting-the implant may be adapted to support various surgical techniques not fully disclosed herein. In various instances, the implant may be applicable for any procedure that may require a biocompatible implant designed to positively impact the quality of life by either treating deformities, stabilizing and strengthening the spine, facilitating fusion, promoting osseointegration, relieving pain, amongst others. Moreover, one benefit of the implant as described herein is the body, surface lattices, rims, edges, inserts, and other features that together the performance of the implant such that implant may better adapt and endure stress, compression, and other mechanical forces of the body.

It will be appreciated by those skilled in the art that while the above disclosure has been described above in connection with particular embodiments and examples, the above disclosure is not necessarily so limited, and that numerous other embodiments, examples, uses, modifications and departures from the embodiments, examples and uses are intended to be encompassed by the claims attached hereto. The entire disclosure of each patent and publication cited herein is incorporated by reference as if each such patent or publication were individually incorporated by reference herein. Various features and advantages of the above disclosure are set forth in the following claims.

We claim:

1. An implant comprising:
a top surface including a first surface lattice;
a bottom surface opposite the top surface, the bottom surface including a second surface lattice; and
a body positioned between the top surface and the bottom surface, the body including a first plurality of lattice structures, wherein the body at least partially defines one or more side surfaces; and
wherein first surface lattice, the second surface lattice, and the body are integrally formed.

2. The implant of claim 1, wherein the first surface lattice, the second surface lattice, and the body are at least partially smooth.

3. The implant of claim 1, wherein the first surface lattice, the second surface lattice, and the body are at least partially coarse.

4. The implant of claim 1 further comprising:
a first surface lattice comprising a second plurality of lattice structures; and
a second surface lattice comprising a third plurality of lattice structures.

5. The implant of claim 1, wherein each lattice structure of the first plurality of lattice structures comprises one or more lattice struts that converge at one or more lattice junctures.

6. The implant of claim 1, wherein the first plurality of lattice structures is formed from a series of repeated lattice structures mirrored over at least one of a vertical axis and a horizontal axis.

7. The implant of claim 1, wherein the implant is symmetrical with respect to at least one of a horizontal axis and a vertical axis.

8. The implant of claim 1, wherein the implant is substantially porous.

9. The implant of claim 1, wherein the implant is configured to be inserted into a spine of a user to provide stability, promote osseointegration, and maintain proper spine alignment.

10. The implant of claim 1, wherein the implant is at least partially formed from at least one of a Titanium alloy and nitinol.

11. An implant to provide stability and promote osseointegration comprising:
a top surface including a first surface lattice;
a bottom surface opposite the top surface, the bottom surface including a second surface lattice;
one or more side surfaces; and
a substantially hollow body including an open framework formed from a plurality of lattice structures, the substantially hollow body disposed between the top surface and the bottom surface, wherein the substantially hollow body at least partially defines the one or more side surfaces; and
wherein the first surface lattice and the second surface lattice at least partially overlay the substantially hollow body about the top surface and the bottom surface, respectively.

12. The implant of claim 11 further comprising:
a first solid perimeter defining an outer periphery of the top surface; and
a second solid perimeter defining an outer periphery of the bottom surface;
wherein the first solid perimeter and the second solid perimeter each bound the first surface lattice and the second surface lattice, respectively.

13. The implant of claim 11, wherein each the first surface lattice and the second surface lattice comprise a plurality of microlattice structures, and wherein the plurality of microlattice structures is substantially thinner than the plurality of lattice structures.

14. The implant of claim 11, wherein the substantially hollow body couples to the first surface lattice and the second surface lattice.

15. The implant of claim 11, wherein the substantially hollow body is integrally formed with the first surface lattice and the second surface lattice.

16. The implant of claim 11, wherein at least one of the one or more side surfaces includes a bore configured to receive a screw.

17. The implant of claim 11 further comprising one or more keels configured to couple the implant to one or more bones.

18. A spinal implant comprising:
a top surface including a first surface lattice;
a bottom surface opposite the top surface, the bottom surface including a second surface lattice;
a substantially hollow body including an open framework formed from a plurality of lattice structures, the substantially hollow body disposed between the top surface and the bottom surface, wherein the substantially hollow body at least partially defines one or more side surfaces, and wherein the first surface lattice and the second surface lattice at least partially overlay the body about the top surface and the bottom surface, respectively; and
an annular channel extending linearly from the top surface to at least a center of the spinal implant, wherein the annular channel defines a space between the top surface and the center of the spinal implant that is free of lattice structures.

19. The implant of claim 18, wherein the annular channel is configured to house a bone graft.

20. The implant of claim 18, wherein the annular channel extends from the top surface to the bottom surface.
